(19) United States
(12) Patent Application Publication
(10) Pub. No.: US 2020/0111542 A1
(43) Pub. Date: Apr. 9, 2020

(54) Method and Device for Analyzing Sequencing Data Result, and Sequencing Library Construction and Sequencing Method

(71) Applicant: China National Rice Research Institute, Hangzhou, Zhejiang (CN)

(72) Inventors: Kejian WANG, Hangzhou, Zhejiang (CN); Qing LIU, Hangzhou, Zhejiang (CN); Chun WANG, Hangzhou, Zhejiang (CN)

(21) Appl. No.: 16/476,079

(22) PCT Filed: May 18, 2018
(86) PCT No.: PCT/CN2018/087509

§ 371 Date: Jul. 4, 2019

(30) Foreign Application Priority Data
Jun. 27, 2017 (CN) 201710504178.3

(51) Int. Cl. G16B 30/00; G16B 20/20; C12Q 1/6874

(52) U.S. Cl. CPC G16B 30/00; C12Q 1/6874; G16B 20/20

(57) ABSTRACT

Provided are a method and device for analyzing a sequencing data result, and a sequencing library construction and sequencing method. The method for analyzing the sequencing data result includes: a sequencing data result of a sequencing library is acquired (S101), the sequencing library including a plurality of mixed samples, each of the samples corresponding to a label sequence combination, and different samples corresponding to different label sequence combinations, wherein each label sequence combination includes a plurality of label sequences, the sequencing data result comprises a sequencing fragment set obtained by sequencing the plurality of mixed samples, and the sequencing fragment set includes a plurality of disordered sequencing fragments; a label sequence combination of each of the sequencing fragments is determined (S102); and a sample corresponding to each of the sequencing fragments is determined according to the label sequence combination of each of the sequencing fragments (S103).

### TECHNICAL FIELD

The present disclosure relates to the field of gene sequencing, and in particular to a method and device for analyzing a sequencing data result, and a sequencing library construction and sequencing method.

### BACKGROUND

As the research on genomics goes deep, there is a growing demand for identifying mutation of sequences of specific areas. Sequence mutation is classified into two types: single base substitution (also called Single Nucleotide Polymorphism, SNP for short) and insertion deletion mutation. The two mutation types are also different. In detection method. The existing methods for identifying SNP mutations include the TaqMan probe method, the SNaPshot method, the Mass Array method, the Illumina BeadXpress method, the Sanger direct sequencing method, the High Resolution Melting (HRM) analysis, and the enzyme digestion method, which can identify both SNP mutations and insertion deletion mutations. Following is a detailed introduction of several methods for identifying SNP mutations.

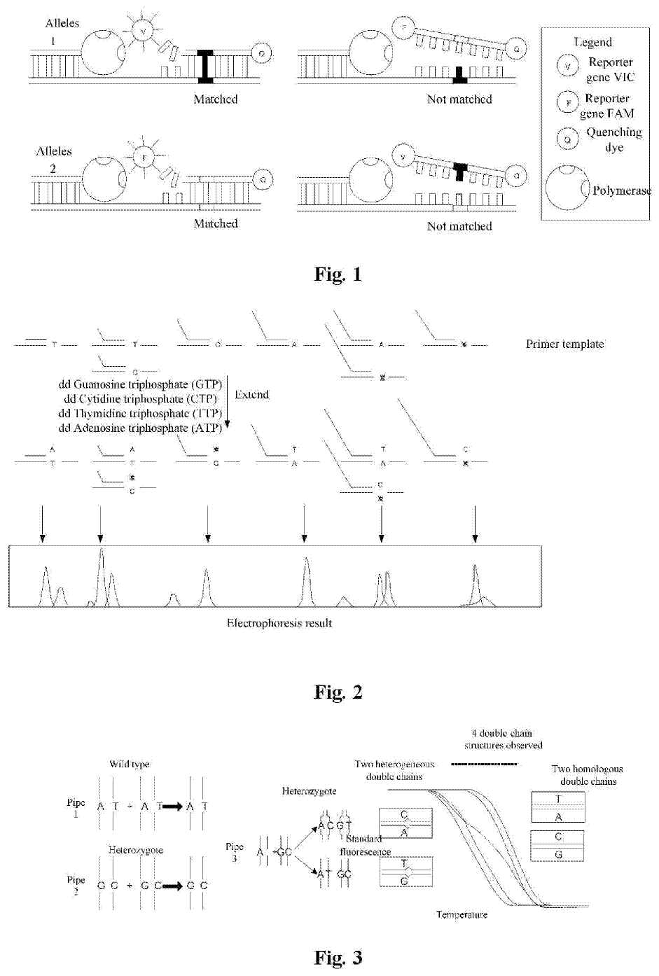

The TaqMan probe method, as shown in FIG. 1, is to design PCR primers and TaqMan probes for different SNP sites on chromosomes, so as to perform real-time fluorescent PCR amplification. A reported fluorescent group and a quenched fluorescent group are labelled at ends 5′ and 3 of the probe respectively. When there is a PCR product in the solution, the probe and the template are annealed to produce a substrate suitable for nucleic acid exonuclease activity, which cuts down from the probe the fluorescent molecule connected at end 5′ of the probe, and destroys the PRET between two fluorescent molecules to emit fluorescence. This method is usually used for analysis of a small number of SNP sites.

As shown in FIG. 2, SNaPshot is a typing technique based on the principle of fluorescent labelling single base extension. It is also called small sequencing, mainly aimed at SNP typing projects with medium throughput. In a reaction system including sequencing enzymes, four fluorescent-labelled ddNTP, different-length extension primers immediately adjacent to end 5′ of the polymorphic site, and a template of PCR products, the primer extends one base and terminates. After detection by an ABI sequencer, the corresponding SNP site of the extended product is determined according to the moving position of the peak, and the types of doped bases are known according to the colour of the peak, thereby the genotype of the sample can be determined. The template of PCR products may be obtained by multiplex PCR reaction systems. It is usually used for analysis of 10-30 SNP sites.

The HRM method, as shown in FIG. 3, is an SNP research tool developed in recent years. It determines whether there is SNP by monitoring in real time the binding between double-stranded DNA fluorescent dyes and PCR amplification products during temperature rising. Moreover, difference in SNP sites, heterozygote, etc. affects the peak shape of the melting curve. Therefore, the HRM analysis can effectively distinguish different SNP sites and different genotypes. This detection method is not limited by mutation base sites and types. Without sequence specific probes, the genotype analysis of samples can be completed by directly running high resolution melting after the end of POR. This method does not need to design probes, features simple and quick operation, low cost, and accurate result, and realizes real closed-tube operation. HRM is a new molecular diagnostic technique combining saturated fluorescent dyes, unlabelled probes and real-time fluorescent quantitative PCR to detect gene mutation and genotyping. The temperature at which half of the DNA double-stranded structure is unlinked is called melting temperature (Tm). Different sequences of DNA correspond to different Tm values. The higher the GC content in DNA is, the higher the Tm value is. The GC content is directly proportional to the Tm value. Non-specific cyanine dyes such as SYBR green can be inserted directly into double-stranded DNA fragments and stimulate fluorescence. Thus, the process of DNA renaturation and denaturation can be shown by fluorescence intensity change in a specific temperature range. The curve formed by the fluorescence signal changing with temperature is the melting curve. Any DNA molecule will have its own melting curve shape and location when it is heated and denatured, mainly because the fragment length, GC content and GC distribution of different nucleic acid molecules are different. For ordinary melting curves, the temperature rises slowly at 0.5 C/cycle. The products of PCR amplification are denatured and the fluorescence signals are detected in real time. Different products will form different characteristic peaks of melting curves. Ordinary Realtime-PCR judges the specificity of amplified products by the specificity of characteristic peaks.

The Mass Array method (also known as the Mass Array molecular weight array technology) is a genetic analysis tool, which enables genotyping detection by combining with the sensitive and reliable MALDI-TOF-MS technology through primer extension or cleavage reaction. The IPLEX GOLD technology based on the Mass Array platform can design up to 40 folds of PCR reaction and genotype detection. The experimental design is flexible and the typing results are accurate. Mass Array has the best cost-effectiveness when hundreds to thousands of samples are tested for dozens to hundreds of SNP loci according to the application needs. It is especially suitable for validating the results of genome-wide research, or for a situation where a limited number of research loci have been identified.

The Illumina BeadXpress method uses a BeadXpress system to detect SNP loci in batches. It can detect 1-384 SNP loci at the same time, and is often used to confirm results of genomic microarray and is suitable for high-throughput detection. Microbead chips have the characteristics of high density, high repeatability, high sensitivity, small sample size, and flexible customization. A high integration density enables a high detection and screening speed, as well as a significant reduction of cost at the time of high-throughput screening.

The mutation identification methods based on the above methods involve the following defects: low throughput, some even merely permitting identification and analysis of a single sample, causing a high cost; low detection efficiency for low-frequency mutation types; Involving complex steps, and requiring biological information background for analysis after obtaining sequencing data.

In the related art, samples of sequencing results need to be identified manually by technicians with technical background, which leads to low efficiency and high cost. With regard to this technical problem, no effective solution has been put forward.

### SUMMARY

Embodiments in the present application provide a method and device for analyzing a sequencing data result and a sequencing library construction and sequencing method to at least solve the technical problem that in the related art, samples of sequencing results need to be identified manually by technicians with technical background, which leads to low efficiency and high cost.

In one aspect, embodiments of the present disclosure provide a method for analyzing a sequencing data result, including: acquiring the sequencing data result of a sequencing library, wherein the sequencing library includes a plurality of mixed samples, each of the samples corresponding to a label sequence combination, and different samples corresponding to different label sequence combinations, and wherein each label sequence combination includes a plurality of label sequences, the sequencing data result includes a sequencing fragment set obtained by sequencing the plurality of mixed samples, the sequencing fragment set including a plurality of disordered sequencing fragments; determining a label sequence combination of each of the sequencing fragments; and determining, according to the label sequence combination of each of the sequencing fragments, a sample corresponding to each of the sequencing fragments.

As at least one alternative embodiment, the plurality of sequencing fragments include a first sequencing fragment, and determining a label sequence combination of the first sequencing fragment includes: extracting all label sequences from the first sequencing fragment; comparing each label sequence extracted from the first sequencing fragment with a plurality of reference label sequences with known numbers, as to determine a corresponding number of each label sequence in the first sequencing fragment; determining a combination of numbers of all label sequences in the first sequencing fragment as a number of the label sequence combination of the first sequencing fragment.

As at least one alternative embodiment, before comparing each label sequence extracted from the first sequencing fragment with the plurality of reference label sequences with known numbers, the method further includes: acquiring the plurality of pre-stored reference label sequences with known numbers.

As at least one alternative embodiment, when the sequencing data result is obtained by a pariend sequencing method, each sequencing fragment includes a forward read sequence and a reverse read sequence; extracting all label sequences from the first sequencing fragment includes: respectively extracting label sequences from the forward read sequence and the reverse read sequence of the first sequencing fragment, wherein the label sequence combination of the first sequencing fragment includes the label sequences extracted from the forward read sequence and the label sequences extracted from the reverse read sequence.

As at least one alternative embodiment, after determining, according to the label sequence combination of each of the sequencing fragments, the sample corresponding to each of the sequencing fragments, the method further includes: acquiring a reference sequence of each sample; extracting sequences of a corresponding sample from each sequencing fragment; comparing the extracted sequences of each corresponding sample with the reference sequence of the corresponding sample, as to determine mutation information of each sample.

As at least one alternative embodiment, acquiring the reference sequence of each sample includes: receiving the reference sequence of each sample, wherein the reference sequence of each sample is uploaded by a client terminal through a control; after determining the mutation information of each sample, the method further includes: feeding back the mutation information of each sample to the client terminal.

As at least one alternative embodiment, acquiring the sequencing data result of the sequencing library includes: receiving the sequencing data result uploaded by a client terminal through a control; after determining, according to the label sequence combination of each of the sequencing fragments, the sample corresponding to each of the sequencing fragments, the method further includes: feeding back a corresponding relationship between the plurality of sequencing fragments and the plurality of samples to the client terminal.

According to another aspect of the present disclosure, a sequencing library construction and sequencing method is provided, including: performing a first round of PCR reaction on a target gene fragment by using a first pair of primers to obtain a first round of PCR product; performing a second round of PCR reaction on the first round of PCR product by using a second pair of primers to obtain a sample, wherein the second pair of primers includes a plurality of label sequences; respectively performing the first round of PCR reaction and the second round of PCR reaction for different target gene fragments to obtain a plurality of samples, wherein different target gene fragments correspond to different label sequence combinations, and the label sequence combination is a combination of multiple label sequences included in the second pair of primers; performing sequencing on the sequencing library to obtain the sequencing data result, wherein the sequencing library includes a plurality of mixtured samples and the sequencing data result includes a plurality of disordered sequencing fragments; performing the method for analyzing a sequencing data result as above on the sequencing data result to obtain an analysis result.

As at least one alternative embodiment, the mixtured multiple samples included in the sequencing library are with equal amounts.

As at least one alternative embodiment, a PCR plate adopted for the second round of PCR reaction is provided with a plurality of holes, each hole for holding a sample, and a number of each hole being a number of the label sequence combination adopted by the sample.

According to an aspect of the present disclosure, a kit is provided, including: a plurality of reagent holes, wherein each reagent hole is provided with a corresponding label, the corresponding label of each reagent hole is configured to indicate a label sequence added to a reagent placed in a corresponding reagent hole.

As at least one alternative embodiment, the kit includes a label plate provided with a plurality of labels, the plurality of labels on the label plate being in one-to-one correspondence with positions of the plurality of reagent holes.

According to another aspect of the present disclosure, a device for analyzing a sequencing data result is provided, including: an acquiring element, configured to acquire the sequencing data result of a sequencing library, wherein the sequencing library includes a plurality of mixed samples, each of the samples corresponding to a label sequence combination, and different samples corresponding to different label sequence combinations, and wherein each label sequence combination includes a plurality of label sequences, the sequencing data result includes a sequencing fragment set obtained by sequencing the plurality of mixed samples, the sequencing fragment set including a plurality of disordered sequencing fragments; a first determining element, configured to determine a label sequence combination of each of the sequencing fragments; and a second determining element, configured to determine, according to the label sequence combination of each of the sequencing fragments, a sample corresponding to each of the sequencing fragments.

In another aspect, embodiments of the present disclosure provide a storage medium including a stored program, wherein a device in which the storage medium is located is controlled to implement the sequencing data result analysis method according to the present disclosure while the program is in operation.

In another aspect, embodiments of the present disclosure provide a processor configured to run the program, wherein the sequencing data result analysis method according to the present disclosure is implemented while the program is in operation.

In embodiments of the present disclosure, a sequencing data result of a sequencing library is acquired, wherein the sequencing library includes a plurality of mixed samples, each of the samples corresponding to a label sequence combination, and different samples corresponding to different label sequence combinations, and wherein each label sequence combination includes a plurality of label sequences, the sequencing data result is a sequencing fragment set obtained by sequencing the plurality of mixed samples, the sequencing fragment set including a plurality of disordered sequencing fragments. A label sequence combination of each of the sequencing fragments is determined, and a sample corresponding to each of the sequencing fragments is determined according to the label sequence combination of each of the sequencing fragments. Through the above embodiment, the technical problem of low efficiency and high cost caused by manually identifying, by technicians with technical background, samples of sequencing results in the related art is solved, and thereby achieving the technical effect of directly determining a sample corresponding to each of the offline data for sequencing that includes multiple mixed samples.

### BRIEF DESCRIPTION OF THE DROWNS

For a better understanding of the present disclosure, accompanying drawings described hereinafter are provided to constitute one part of the application; the schematic embodiments of the present disclosure and the description thereof are used to illustrate the present disclosure but to limit the present disclosure improperly. In the accompanying drawings:

FIG. 1 is a schematic diagram of detecting SNP in a TaqMan probe method in the related art;

FIG. 2 is a schematic diagram of detecting SNP in a SNaPshot method in the related art;

FIG. 3 is a schematic diagram of detecting SNP in an HRM technique in the related art;

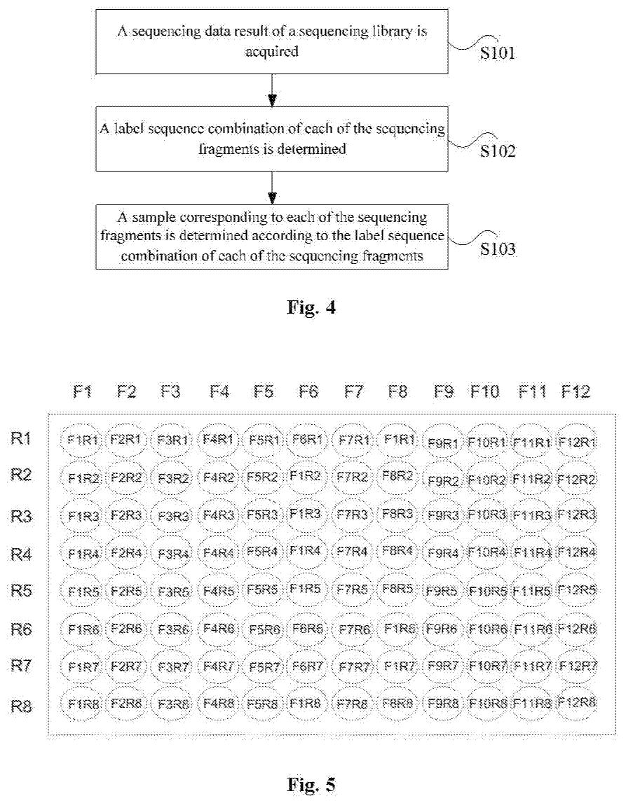

FIG. 4 is a flow chart of an optional method for analyzing the sequencing data result according to embodiments of the present disclosure;

FIG. 5 is a schematic diagram of an optional barcode panel according to embodiments of the present disclosure;

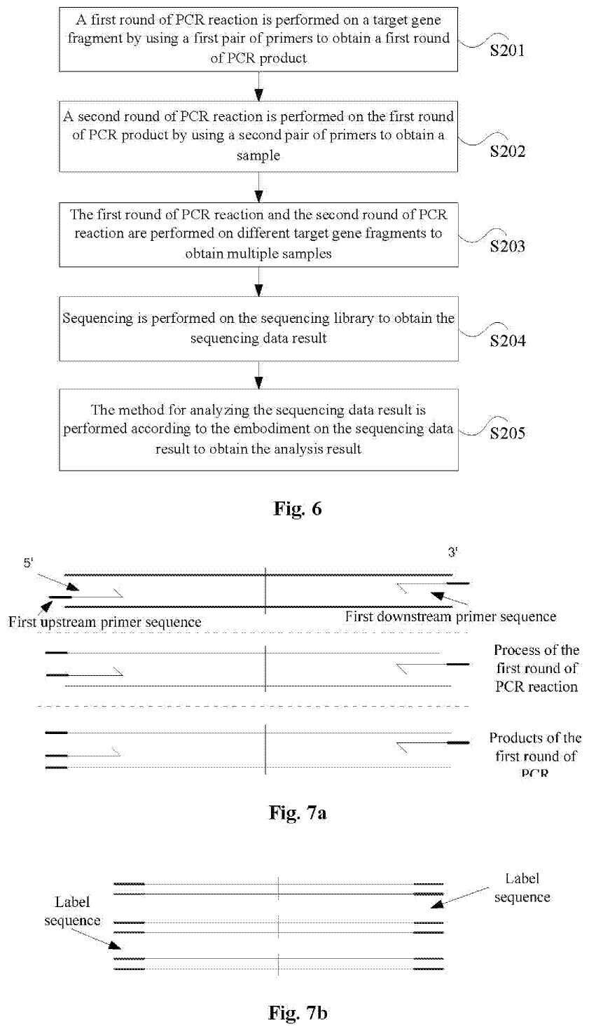

FIG. 6 is a flow chart of an optional sequencing library construction and sequencing method according to embodiments of the present disclosure;

FIG. 7a is a schematic diagram of an optional first round of amplification according to embodiments of the present disclosure;

FIG. 7b is a schematic diagram of products of an optional first round of amplification according to embodiments of the present disclosure; and

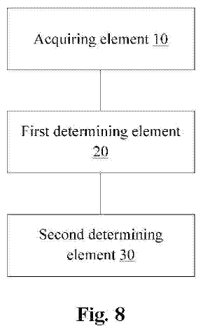

FIG. 8 is a schematic diagram of an optional device for analyzing the sequencing data result according to embodiments of the present disclosure.

### DETAILED DESCRIPTION OF THE EMBODIMENTS

To make those skilled in the art better understand the technical solutions of the present disclosure, the technical solutions in the embodiments of the present disclosure will be described clearly and completely with reference to the accompanying drawings in the embodiments of the present disclosure. Obviously, the described embodiments are merely some but not all of embodiments of the present disclosure. All other embodiments made on the basis of the embodiments of the present disclosure by a person of ordinary skill in the art without paying any creative effort shall be included in the protection scope of the present disclosure.

It should be noted that the terms “first”, “second” and the like in the description, claims and the drawings of the present disclosure are used to distinguish similar objects rather than to describe a specific sequence. It should be understood that the data used in this way are interchangeable in appropriate cases, so that the embodiments of the present disclosure described herein can be implemented in a sequence other than those illustrated or described herein. In addition, the terms “include” and “include” and any variations thereof are intended to cover non-exclusive inclusions, e.g. a process, a method, a system, a product or a device that includes a series of steps or units includes not only the steps or units clearly listed, but also those not clearly listed, and other steps or units that are inherent to the process, method, product or device.

The present application provides embodiments of a method for analyzing a sequencing data result.

FIG. 4 is a flow chart of an optional method for analyzing the sequencing data result according to embodiments of the present disclosure. As shown in FIG. 4, the method includes:

Step S101: the sequencing data result of a sequencing library is acquired;

Step S102: a label sequence combination of each of the sequencing fragments is determined;

Step S103: a sample corresponding to each of the sequencing fragments is determined according to the label sequence combination of each of the sequencing fragments.

The sequencing library is a pre-constructed gene library. A sequencing library includes multiple mixed samples, and each of the samples may be obtained by processing a target gene fragment, wherein the target gene fragment refers to the gene fragment that need to be tested (for example, a mutation identification test). However, due to the need for sequencing the mixture of multiple genes, it is impossible to distinguish between the target gene fragments in the sequencing results. Therefore, each target gene fragment needs to be processed. At least a label sequence for marking should be added to the target gene fragment to obtain a sample, so that each sample can be distinguished from other samples. Therefore, each sample corresponds to a label sequence combination, and different samples correspond to different label sequence combinations, wherein each label sequence combination includes a plurality of label sequences, the sequencing data result includes a sequencing fragment set obtained by sequencing the plurality of mixed samples, the sequencing fragment set includes a plurality of disordered sequencing fragments.

As at least one alternative embodiment, the sequencing library may be constructed by a general library construction method. For example, multiple samples may be obtained by two rounds of PCR amplification respectively on the multiple target gene fragments (the target gene fragment refers to one fragment of a gene, and multiple target gene fragments may be the same fragment of a gene in different sample objects). The primers used for PCR amplification include multiple label sequences, which are used as markers of target gene fragments. Each sample in the sequencing library includes multiple label sequences, and the combinations of multiple label sequences of each sample are different.

For example, the multiple label sequences include R1, R2, F1, F2 and so on. Each target gene fragment is labelled by two label sequences. The first target gene fragment is labelled by R1 and F1, the second target gene fragment is labelled by R1 and F2, the third target gene fragment is labelled by R2 and F1, and the fourth target gene fragment is labelled by R2 and F2. In other words, the label sequence combinations of each target gene fragment are different. The above examples of label sequence combinations for labelling different target gene fragments are merely illustrative and do not constitute a limitation to the technical solution of the present application.

After obtaining the sequencing library, the sequencing library may be sequenced through a sequencing platform to get the sequencing off-line data, that is, the sequencing data results. Because the multiple samples in the sequencing library are mixed in the sequencing process, the sequencing fragments in the sequencing data results are disordered, with each sequencing fragment corresponding to a sample, but it is unknown which sample is corresponding to which sequencing fragment. Therefore, after the sequencing data results of the sequencing library are obtained, the label sequence combinations of each sequencing fragment are determined, and a one-to-one correspondence is established between the multiple sequencing fragments and the multiple samples according to the label sequence combinations of each sequencing fragment.

It should be noted that the data processing method provided in the embodiments is executed by software, in particular, by programs or applications installed on the terminal device. As at least one alternative embodiment, the embodiments may be executed by a server. When acquiring a sequencing data result of a sequencing library, the server may receive the sequencing data result uploaded by the client terminal through the control (e.g. an input box on a web page); after establishing a one-to-one correspondence between the multiple sequencing fragments and the multiple samples according to the label sequence combination of each of the sequencing fragments, the server feeds back the correspondence between the multiple sequencing fragments and the multiple samples to the client terminal.

For example, the data processing method provided in the embodiments may be performed by a server. In step S101, the sequencing data results may be obtained by receiving, by the server, the sequencing data results sent over the network by the request terminal (another terminal requesting data processing on the sequencing data results), and in step S103, the server may obtain the correspondence between each sample in the sequencing library and each sequencing fragment in the sequencing data result, and then send the correspondence to the request terminal over the network. Further, the server may obtain the reference sequence of each target gene fragment (sample sequence) uploaded by the request terminal through the network and, after comparing the sample sequence in each sequence fragment with the corresponding reference sequence, feed back the mutation identification result to the request terminal.

The server may receive data sent by the request terminal through a web page. The server may use Linux system and Apeche software. The database may be the Mysql (MariaDB, for example) database system. The web page may be built by using Perl, PHP or Python language scripts. For example, programs in the server may include Perl scripts combined with shell execution scripts, and the website analysis interface may be built by PHP language combined with JavaScript language.

In an optional embodiment, taking one of the multiple sequencing fragments (first sequencing fragment) included in the sequencing data results as an example, that a label sequence combination of the first sequencing fragment is determined may includes: all label sequences are extracted from the first sequencing fragment; each label sequence extracted from the first sequencing fragment is compared with a plurality of reference label sequences with known numbers to determine a corresponding number of each label sequence in the first sequencing fragment; a combination of numbers of all label sequences in the first sequencing fragment is determined as a number of a label sequence combination in the first sequencing fragment. Each sequencing fragment contains at least multiple label sequences and sample sequences (sequencing results of target gene fragments).

As at least one alternative embodiment, the sequencing data result may exist in the format of data compression package. When acquiring the sequencing data result of the sequencing library, the data compression package is decompressed and multiple sequencing fragments may be obtained. For example, each sequencing fragment may exist in the form of a data packet, each data packet may include multiple segments of sequencing data, each segment may be a sample sequence, and a label sequence or one of the other sequences may include in the sample. The label sequences are extracted from the data packet of the first sequencing fragment and compared with the reference label sequences with known numbers. The reference label sequences with known numbers are determined by the construction method of sequencing library. The label sequences extracted from the data packet are compared with the label sequence library used in the construction of sequencing library to determine the numbers of the label sequences extracted from the data packet.

Before each label sequence extracted from the first sequencing fragment is compared with the plurality of reference label sequences with known numbers, the pre-stored plurality of reference label sequences with known numbers may be need to be acquired. As at least one alternative embodiment, the plurality of reference label sequences with known numbers may be data uploaded by the client or data pre-stored locally by the server.

As at least one alternative embodiment, in the case that the sequencing data result is obtained by a pariend sequencing method, each sequencing fragment includes a forward read sequence and a reverse read sequence; that all label sequences are extracted from the first sequencing fragment may include: label sequences are extracted from the forward read sequence and the reverse read sequence of the first sequencing fragment, respectively, wherein the label sequence combination of the first sequencing fragment includes label sequences extracted from the forward read sequence and label sequences extracted from the reverse read sequence.

Establishing a one-to-one correspondence between the multiple sequencing fragments and the multiple samples according to the label sequence combination of each of the sequencing fragments may include: the sample corresponding to the first sequencing fragment is determined according to the corresponding number of each label sequence in the first sequencing fragment, and the sample corresponding to each sequencing fragment is determined by using the same processing method as the first sequencing fragment.

After determining the sample corresponding to each sequencing fragment according to the label sequence combination of each of the sequencing fragments, the method may further include that: a reference sequence of each sample is acquired; sequences of samples from each sequencing fragment are extracted; each extracted sample sequence is compared with a reference sequence of a corresponding sample to determine mutation information of each sample.

Further, the step of acquiring the reference sequence of each sample may include that: a reference sequence of each sample uploaded by a client terminal through a control is received; after determining mutation information of each sample, the method further includes: the mutation information of each sample is feed back to the client terminal.

Similarly, the step of acquiring the sequencing data result of the sequencing library may include that: the sequencing data result uploaded by the client terminal through the control is received; after determining, according to the label sequence combination of each of the sequencing fragments, a sample corresponding to each of the sequencing fragments, the method further includes: a correspondence between the plurality of sequencing fragments and the plurality of samples is feed back to the client terminal.

In this embodiment, the sequencing data result of the sequencing library is acquired, wherein the sequencing library includes a plurality of mixed samples, each of the samples corresponding to a label sequence combination, and different samples corresponding to different label sequence combinations, and wherein each label sequence combination includes a plurality of label sequences, the sequencing data result includes a sequencing fragment set obtained by sequencing the plurality of mixed samples, the sequencing fragment set including a plurality of disordered sequencing fragments. A label sequence combination of each of the sequencing fragments is determined, and a sample corresponding to each of the sequencing fragments is determined according to the label sequence combination of each of the sequencing fragments. Through the above solution, the technical problem of low efficiency and high cost caused by manually identifying, by technicians with technical background, samples of sequencing results in the related art is solved, thereby achieving the technical effect of directly determining a sample corresponding to each of the offline data to be sequenced that includes multiple mixed samples.

The data processing method provided in this embodiment can improve the efficiency of mutation identification, and high-throughput analysis results can be obtained without a biological information background. Further, on the basis of identifying the corresponding data of each sample, the sequencing result of each sample is compared with the reference gene, and the mutation identification result is obtained. This is a new high-throughput mutation identification method, which can simplify the experimental steps.

In an optional embodiment, the data processing method in an optional application scenario is described, including:

Step 1: the request terminal uploads the sequencing result data (in the format of a compressed package) to the server (through PHP);

Step 2: the server calls local decompression software (e.g. gunzip) to decompress the uploaded data;

Step 3: the server (using Perl script) extracts the barcode (label sequence) combination of each pairend sequence (double-ended sequencing sequence);

Step 4: the server (using Perl script) combines with the barcode combination to determine the number of each sample in the sequencing library.

As at least one alternative embodiment, the sequencing library may be placed on a test orifice plate (or called barcode plate, label sequence plate) as shown in FIG. 5. Each orifice on the test orifice plate corresponds to one label sequence combination, and the label sequence combinations are different from each other. As shown in FIG. 5, the label sequences include 20 kinds of labels, including F1-F12 and R1-R8, and they form 12*8=96 label sequence combinations (F1R1, F2R1, etc.). Each label sequence combination corresponds to a hole in the orifice plate, and one sample is placed in each hole. The test orifice plate may be placed directly in the sequencing instrument for sequencing.

Therefore, when the server obtains the database of all the label sequences and knows the above 20 kinds of labels, i.e., F1-F12 and R1-R8, the server may compare the label sequence of each sequencing fragment in the sequencing data result with the known label sequence, and determines the label sequence combination (such as F1R1 and F2R1) of each sequencing fragment. After determining the label sequence combination of each sequencing fragment, the server may determine the number of each sample (for example, the sample number may also be labelled by label sequence combination), and also may determine the correspondence between each sequencing fragment in the sequencing data result and each hole on the test orifice plate. As at least one alternative embodiment, the database of all label sequences may be uploaded by the request terminal or may be a universal label sequence database pre-stored in the database of the server.

Step 5: the server (using a local short-sequence data comparison tool, e.g. BWA software) compares each sample sequence with the reference genome sequence uploaded by the request terminal.

As at least one alternative embodiment, before Step 5, the server needs to acquire the reference genome sequence uploaded by the request terminal, and the step of acquiring the reference genome sequence uploaded by the request terminal may be available as long as it is executed before the step 5. In other words, merely after the server receives the reference genome sequence, this step 5 may be executed.

Step 6: the server (using Perl script) analyses, collates and counts the mutation information of each sample.

Step 7: the request terminal downloads the analysis results of the server (through PHP).

It should be noted that although the flowchart in the accompanying drawings shows a logical order, in some cases, the steps shown or described may be performed in a different order.

The present application further provides an embodiment for a storage medium, the storage medium including a stored program, wherein a device in which the storage medium is located is controlled to implement the method for analyzing the sequencing data result according to the embodiment of the present disclosure while the program is in operation.

The present application further provides an embodiment for a processor configured to run the program, wherein the method for analyzing the sequencing data result according to the embodiment of the present disclosure is implemented while the program is in operation.

The present application further provides an embodiment for a sequencing library construction and sequencing method.

FIG. 6 is a flow chart of an optional sequencing library construction and sequencing method according to embodiments of the present disclosure. As shown in FIG. 6, the method includes:

Step S201: a first round of PCR reaction is performed on a target gene fragment by using a first pair of primers to obtain a first round of PCR product.

As at least one alternative embodiment, the target region fragments (target gene fragments) of different sample materials may be amplified and enriched through a first pair of primers by integrating the bridging sequence and the specific primer sequence for target fragment amplification on the first pair of primers amplified on upstream and downstream. It should be noted that before this step, there is a step of extracting genomic DNA (genes) from the sample to be tested. DNA samples extracted by any method are acceptable, and there is no special requirement for the amount and concentration of the sample.

As shown in FIG. 7a, the first pair of primers includes the first upstream primer sequence and the first downstream primer sequence. The solution of the first upstream primer sequence and the first downstream primer sequence are mixed with the solution of DNA sequence of a single sample, and a first PCR product is obtained from the PCR reaction. As at least one alternative embodiment, the first upstream primer sequence or the first downstream primer sequence may sequentially include the bridging sequence and the specific primer sequence for the first upstream target fragment amplification or the specific primer sequence for the first downstream target fragment amplification from end 5′ to end 3′. The bridging sequence is complementary to and paired with the primer sequence of the second round of PCR, and the specific primer sequence for the first upstream target fragment amplification and the specific primer sequence for the first downstream target fragment amplification complement the end 3′ of two single strands of DNA sequence respectively. The length of the specific primer sequence for target fragment amplification may be 15-25 BP, and the length of the bridging sequence may be 15-30 bp. The first upstream primer sequence and the first downstream primer sequence may also be added to the label sequence, and the length of the label sequences added is random, 1-50 bp maybe, then by random combination, a large number of samples may be distinguished at once.

Step S202: a second round of PCR reaction is performed on the first round of PCR product by using a second pair of primers to obtain a sample.

The second pair of primers includes multiple label sequences, a different target gene fragment corresponds to a different label sequence combination, and the label sequence combination may be a combination of multiple label sequences included in the second pair of primers. The second round of PCR products are shown in FIG. 7b. Each sample has a label sequence on each of the two ends.

As at least one alternative embodiment, the joint sequence, the sequencing primer, the label sequence and the bridging sequence may be integrated into a universal second pair of primers. The second round of primers, being a combination of universal primers, have been arranged into a fixed combination illustrated in a 96-hole plate (as shown in FIG. 5) or in a 384-hole plate, thereby a mixed kit is made. The second pair of primers include the second upstream primer sequence and the second downstream primer sequence. The PCR product obtained in step S201 is mixed with different combinations of solutions of the second upstream primer sequence and the second downstream primer sequence respectively. Alternatively, the second PCR product may be obtained from a PCR reaction by directly using the configured mixed kit (e.g. the 96-hole or 384-hole kit for mixing the second-round primer). As at least one alternative embodiment, end 5′ to end 3′ of the second upstream primer sequence may sequentially include the joint sequence, the sequencing primer sequence, the label sequence and the bridging sequence of the first upstream primer sequence. End 5′ to end 3 of the second downstream primer sequence may sequentially include the joint sequence, the sequencing primer sequence, the label sequence, and the bridging sequence of the first downstream primer sequence. Label sequences of each pair of the second upstream primer sequence and the second downstream primer sequence enable each DNA sequence to have a label sequence different from those of other DNA sequences after PCR reaction. The length of the label sequence may be 1 to 20 bp. By virtue of pairend sequencing (double-end sequencing) and in combination with the label sequences on both sides, mixing of 1 to +∞ samples may be distinguished simultaneously.

As at least one alternative embodiment, 4 to 10 bases may be introduced between the sequencing primer sequence and the label sequence to improve the accuracy of the label sequence arising from the sequencing.

Step S203: the first and second rounds of PCR reactions are performed on different target gene fragments to obtain multiple samples.

It should be noted that a different target gene fragment corresponds to a different label sequence combination, and the label sequence combination is a combination of multiple label sequences included in the second pair of primers.

As at least one alternative embodiment, the mixed multiple samples included in the sequencing library are equal amounts. Two rounds of PCR reactions are performed on different target samples, and the second round of PCR products mixed by equal amounts are purified. A sequencing library may be obtained, which includes multiple mixed samples.

The cost of synthetic primers can be saved by the above two rounds of amplification. In addition, with this method, the construction of sequencing library can be completed by merely two steps of amplification. This not only improves the quality of the sequencing library and the library-building efficiency, but also makes the reagents used for conventional on-line sequencing available to be used to the high-throughput sequencing on the built sequencing library, without a need to provide additional sequencing primers and change the sequencing primers mixed into the MiX library, because the sequencing library has the joint sequence and sequencing primer sequence used on conventional sequencing platforms.

Step S204: Sequencing is performed on the sequencing library to obtain the sequencing data result.

As at least one alternative embodiment, the sequencing library includes multiple mixed samples, and a high-throughput sequencing platform may be used for second-generation sequencing of the sequencing library to obtain sequencing off-line data, that is, sequencing data results, wherein the sequencing data result includes multiple disordered sequencing fragments. As at least one alternative embodiment, after obtaining the sequencing library and before high-throughput sequencing, there are also steps for quality control of the sequencing library.

Step S205: The method for analyzing the sequencing data result according to the above embodiment is performed on the sequencing data result to obtain the analysis result.

By applying the sequencing method provided in the embodiment and through two rounds of PCR and second-generation sequencing, the sequencing off-line data may be directly processed by a data processing method, which can realize the effect of automatic identification of the sequencing data result of the multiple mixed samples. It should be noted that the number of mixed samples may be adjusted by adjusting the number of combinations of the second round of primer pairs. As at least one alternative embodiment, after the sequencing data result is identified, mutations in the target gene fragment of each sample may be identified and analyzed automatically.

As at least one alternative embodiment, the label sequence in the primer sequence adopted in step S202 is used preferably to distinguish the samples. When the discrimination fails, the identification sequence provided by a sequencing company may be used to distinguish multiple DNA sequences from different samples.

As at least one alternative embodiment, a PCR plate adopted for the second round of PCR reaction is provided with a plurality of holes, each hole for holding a sample, and the number of each hole being the number of the label sequence combination adopted for the sample.

In the sequencing method provided in the embodiment, mutation information is identified by a library building method for high-throughput amplicon mutation identification and the analysis software corresponding to the library building method. Compared with other mutation identification methods, this sequencing method has new improvements in library building and analysis methods. The sequencing method also has automatic decoding and mutation identification software, featuring lower cost, shorter library building time, and simpler operation, with the mixed samples distinguished at one time not limited in number. In this method, the sequencing result of mixed samples can be automatically split and the mutation type of the individual material can be automatically identified. This operation method is simple and can be used to identify a large number of samples without any bioinformatic background.

The present application further provides an embodiment for a kit, the kit including a plurality of reagent holes, wherein each reagent hole is provided with a corresponding label configured to indicate a label sequence added to the reagent placed in the corresponding reagent hole. As at least one alternative embodiment, the labels may be arranged on a label plate. For example, the kit may include a label plate which is provided with a plurality of labels by means of gluing, printing, etc., the plurality of labels on the label plate being in one-to-one correspondence with positions of the plurality of reagent holes.

For example, the kit provided in the embodiment may include a barcode (label) plate as shown in FIG. 5. Each label on the barcode plate corresponds to a reagent hole, and the label of each reagent hole indicates the number of a reagent hole. The reagent corresponding to each reagent hole may be added into two label sequences, and the number of each label includes the number of the two label sequences added. At most 96 reagents may be labelled by different label sequences formed by 20 numbers as shown in FIG. 5.

The present application further provides embodiments of a device for analyzing a sequencing data result.

FIG. 8 is a schematic diagram of an optional device for analyzing the sequencing data result according to embodiments of the present disclosure. As shown in FIG. 8, the device includes: an acquiring element 10, a first determining element 20, and a second determining element 30, wherein the acquiring element 10 is configured to acquire a sequencing data result of a sequencing library, wherein the sequencing library includes a plurality of mixed samples, each of the samples corresponding to a label sequence combination, and different samples corresponding to different label sequence combinations, and wherein each label sequence combination includes a plurality of label sequences, the sequencing data result includes a sequencing fragment set obtained by sequencing the plurality of mixed samples, the sequencing fragment set including a plurality of disordered sequencing fragments; the first determining element 20 is configured to determine a label sequence combination of each of the sequencing fragments; and the second determining element 30 is configured to determine, according to the label sequence combination of each of the sequencing fragments, a sample corresponding to each of the sequencing fragments.

In this embodiment, the sequencing data result of the sequencing library is acquired by the acquiring element 10, wherein the sequencing library includes a plurality of mixed samples, each of the samples corresponding to a label sequence combination, and different samples corresponding to different label sequence combinations, and wherein each label sequence combination includes a plurality of label sequences, the sequencing data result includes a sequencing fragment set obtained by sequencing the plurality of mixed samples, the sequencing fragment set including a plurality of disordered sequencing fragments. The label sequence combination of each of the sequencing fragments is determined by the first determining element 20, and the sample corresponding to each of the sequencing fragments is determined by the second determining element 30 according to the label sequence combination of each of the sequencing fragments. Through the above embodiment, the technical problem of low efficiency and high cost caused by manually identifying, by technicians with technical background, samples of sequencing results in the related art is solved, and thereby achieving the technical effect of directly determining a sample corresponding to each of the offline data to be sequenced that includes multiple mixed samples.

In an optional implementation method of the above embodiment, the plurality of sequencing fragments includes a first sequencing fragment, and the first determining element 20 includes: an extracting component configured to extract all label sequences in the first sequencing fragment; and a comparing component configured to compare the extracted multiple label sequences with a plurality of reference label sequences with known numbers, to determine the number corresponding to each label sequence in the first sequencing fragment.

It should be noted here that the acquiring element 10, the first determining element 20 and the second determining element 30 may run in a computer terminal as a part of the device. The functions of the above components may be implemented by the processor in the computer terminal. The computer terminal may be a smart phone (Android mobile phone, iOS mobile phone, etc.), a tablet computer, a palm computer, a mobile Internet device (MID), PAD, etc.

The above device may include a processor and a memory, the above elements may be stored in the memory as a program element, and the processor executes the program element stored in the memory to achieve the corresponding functions.

Memories may include non-permanent memory, random access memory (RAM) and/or non-volatile memory in computer-readable media, such as read-only memory (ROM) or flash memory (flash RAM). A memory includes at least one memory chip.

The sequence of the above-mentioned embodiments in the present application is irrelevant to superiority or inferiority of the embodiments.

In the above-mentioned embodiments of the present application, each embodiment is emphasized in some aspect, and for a part not detailed in one embodiment, reference may be made to relevant descriptions in other embodiments. In the embodiments provided in the present application, it should be understood that the disclosed technical contents may be implemented in other ways.

The device embodiments described above are merely illustrative. For example, the elements may be divided by logical function, but in implementation, there may be another division method. For example, multiple elements or components may be combined or integrated into another system, or some features may be ignored or not executed. Another point is that the coupling, direct coupling or communication connection shown or discussed may be indirect coupling or communication connection via some interfaces, elements or components, and may be electrical or in other forms.

In addition, the functional elements in the embodiments of the present application may be integrated in one processing element, may be physically present separately, or two or more of them are integrated in one element. The above integrated elements may be implemented in the form of hardware or as software function elements.

The integrated elements can be stored in a computer readable storage medium when they are implemented as software functional elements and sold or used as independent products. Based on this understanding, the technical solution of the present application can be embodied as a software product in essence, or as a whole or in part, or for the part contributing to the related art. The computer software product is stored in a storage medium, including several instructions to enable a computer (a personal computer, a server, a network device, etc.) to implement all or part of the steps of the method described in the embodiments of the present application. The storage media mentioned above include U disk, Read-Only Memory (ROM), Random Access Memory (RAM), Mobile Hard Disk, Disk, CD, and the like that can store program codes.

The above are merely the example embodiments of the present disclosure and not intended to limit the present disclosure. For those skilled in the art, various modifications and changes can be made to the present disclosure. Any modification, equivalent substitution and improvement made within the spirit and principle of the present disclosure are intended to be included within the scope of protection of the present disclosure.

What is claimed is:

1. A method for analyzing a sequencing data result, comprising:
acquiring the sequencing data result of a sequencing library, wherein the sequencing library comprises a plurality of mixed samples, each of the samples corresponding to a label sequence combination, and different samples corresponding to different label sequence combinations, and wherein each label sequence combination comprises a plurality of label sequences, the sequencing data result comprises a sequencing fragment set obtained by sequencing the plurality of mixed samples, the sequencing fragment set comprising a plurality of disordered sequencing fragments;
determining a label sequence combination of each of the sequencing fragments; and
determining, according to the label sequence combination of each of the sequencing fragments, a sample corresponding to each of the sequencing fragments.

2. The method as claimed in claim 1, wherein the plurality of sequencing fragments comprise a first sequencing fragment, and determining a label sequence combination of the first sequencing fragment comprises:
extracting all label sequences from the first sequencing fragment;
comparing each label sequence extracted from the first sequencing fragment with a plurality of reference label sequences with known numbers, as to determine a corresponding number of each label sequence in the first sequencing fragment;
determining a combination of numbers of all label sequences in the first sequencing fragment as a number of the label sequence combination of the first sequencing fragment.

3. The method as claimed in claim 2, wherein before comparing each label sequence extracted from the first sequencing fragment with the plurality of reference label sequences with known numbers, the method further comprises:
acquiring the plurality of pre-stored reference label sequences with known numbers.

4. The method as claimed in claim 2, wherein when the sequencing data result is obtained by a pariend sequencing method, each sequencing fragment comprises a forward read sequence and a reverse read sequence; extracting all label sequences from the first sequencing fragment comprises:
respectively extracting label sequences from the forward read sequence and the reverse read sequence of the first sequencing fragment, wherein the label sequence combination of the first sequencing fragment comprises the label sequences extracted from the forward read sequence and the label sequences extracted from the reverse read sequence.

5. The method as claimed in claim 1, wherein after determining, according to the label sequence combination of each of the sequencing fragments, the sample corresponding to each of the sequencing fragments, the method further comprises:
acquiring a reference sequence of each sample;
extracting sequences of a corresponding sample from each sequencing fragment;
comparing the extracted sequences of each corresponding sample with the reference sequence of the corresponding sample, as to determine mutation information of each sample.

6. The method as claimed in claim 5, wherein
acquiring the reference sequence of each sample comprises: receiving the reference sequence of each sample, wherein the reference sequence of each sample is uploaded by a client terminal through a control;
after determining the mutation information of each sample, the method further comprises: feeding back the mutation information of each sample to the client terminal.

7. The method as claimed in claim 1, wherein
acquiring the sequencing data result of the sequencing library comprises: receiving the sequencing data result uploaded by a client terminal through a control;
after determining, according to the label sequence combination of each of the sequencing fragments, the sample corresponding to each of the sequencing fragments, the method further comprises: feeding back a corresponding relationship between the plurality of sequencing fragments and the plurality of samples to the client terminal.

8. A sequencing library construction and sequencing method, comprising:
performing a first round of PCR reaction on a target gene fragment by using a first pair of primers to obtain a first round of PCR product;
performing a second round of PCR reaction on the first round of PCR product by using a second pair of primers to obtain a sample, wherein the second pair of primers comprises a plurality of label sequences;
respectively performing the first round of PCR reaction and the second round of PCR reaction for different target gene fragments to obtain a plurality of samples, wherein different target gene fragments correspond to different label sequence combinations, and the label sequence combination is a combination of multiple label sequences comprised in the second pair of primers;
performing sequencing on the sequencing library to obtain the sequencing data result, wherein the sequencing library comprises a plurality of mixtured samples and the sequencing data result comprises a plurality of disordered sequencing fragments;
performing the method for analyzing a sequencing data result as claimed in claim 1 on the sequencing data result to obtain an analysis result.

9. The method as claimed in claim 8, wherein the mixtured multiple samples comprised in the sequencing library are with equal amounts.

10. The method as claimed in claim 8, wherein a PCR plate adopted for the second round of PCR reaction is provided with a plurality of holes, each hole for holding a sample, and a number of each hole being a number of the label sequence combination adopted by the sample.

11. A kit, comprising:
a plurality of reagent holes, wherein each reagent hole is provided with a corresponding label, the corresponding label of each reagent hole is configured to indicate a label sequence added to a reagent placed in a corresponding reagent hole.

12. The kit as claimed in claim 11, wherein the kit comprises a label plate provided with a plurality of labels, the plurality of labels on the label plate being in one-to-one correspondence with positions of the plurality of reagent holes.

13-15. (canceled)
